(19) United States
(12) Patent Application Publication
(10) Pub. No.: US 2023/0128100 A1
(43) Pub. Date: Apr. 27, 2023

(54) HEALTHCARE INFORMATION PROCESSING APPARATUS AND HEALTHCARE INFORMATION SERVER

(71) Applicant: CANON MEDICAL SYSTEMS CORPORATION, Otawara-shi (JP)

(72) Inventors: Mariko SHIBATA, Nasushiobara (JP); Katsuhiko FUJIMOTO, Saitama (JP); Michitaka SUGAWARA, Utsunomiya (JP)

(73) Assignee: CANON MEDICAL SYSTEMS CORPORATION, Otawara-shi (JP)

(21) Appl. No.: 17/937,874

(22) Filed: Oct. 4, 2022

(30) Foreign Application Priority Data
Oct. 22, 2021 (JP) 2021-173517

(51) Int. Cl. G16H 10/60; G16H 50/20

(52) U.S. Cl. CPC G16H 10/60; G16H 50/20

(57) ABSTRACT

The healthcare information processing apparatus according to the present embodiment comprises a memory; and processing circuitry configured to collect at least one information among a vital information, an activity information, and an environment information of a subject, associate with a date and/or time, and store in a memory; ask questions to the subject when the information meets a predefined condition based on the information of the subject; and cause the memory to store a response to the questions.

### CROSS REFERENCE TO RELATED APPLICATIONS

This application is based upon and claims the benefit of priority from the prior Japanese Patent Application No. 2021-173517, filed on Oct. 22, 2021, the entire contents of which are incorporated herein by reference.

### FIELD

The embodiments disclosed in the present specification and drawings relate to a healthcare information processing apparatus and a healthcare information server.

### BACKGROUND

For medical workers such as a doctor to correctly input a medical chart at the time of a medical interview with less labor, a medical interview form input system in which a patient answers the medical interview form online from home etc. before visiting, is developed. Likewise, to make the patient's response to the medical interview easier, an electronic medical chart input support apparatus which allows voice input is also developed. Furthermore, a web medical interview system using an Artificial Intelligence (AI) to optimize questions for each patient to improve efficiency of medical workers filling out medical charts, is also commercialized.

Also, in the conventional way of doing medical interviews by responding to a question of the doctor, the patient may get nervous in front of the doctor and may respond to the medical interview without thinking properly. For this reason, by utilizing various technologies as described above, the medical interview is also performed in advance, but if the patient lacks medical knowledge, the response may be incorrect even if there is enough time to think. Likewise, the patient may not remember when the symptom started, a time or timing which the symptom occurs well, or an occurrence frequency of symptom. For this reason, even if the words of the patient are properly recorded on the medical chart with voice input etc., the medical interview results may not be always accurate. However, since the medical interview result is greatly relevant to the diagnosis or treatment plans thereafter, it is desirable to be comprehensive and accurate as possible.

### BRIEF DESCRIPTION OF THE DRAWINGS

FIG. 1 is a block diagram describing an example of an entire configuration of a healthcare information processing system according to a first embodiment.

FIG. 2 is a diagram illustrating in a list, an exemplary sensor which a user information collecting apparatus in the healthcare information processing system shown in FIG. 1 may comprise.

FIG. 3 is a block diagram illustrating an example of an inner configuration of the healthcare information processing apparatus in the healthcare information processing system shown in FIG. 1.

FIG. 4 is a diagram illustrating a flowchart describing contents of the user information collecting process executed by the healthcare information processing apparatus in the healthcare information processing system shown in FIG. 1.

FIG. 5 is a diagram illustrating a flowchart describing the contents of a medical interview data generating process executed by the healthcare information processing apparatus in the healthcare information processing system shown in FIG. 1.

FIG. 6 is a diagram illustrating an example of a collected data held by the healthcare information processing apparatus according to the present embodiment.

FIG. 7 is a diagram illustrating an exemplary state in which a clinical department candidate data is added to the collected data held by the healthcare information processing apparatus according to the present embodiment.

FIG. 8 is a diagram illustrating an example of the medical interview data generated by the healthcare information processing apparatus according to the present embodiment based on the medical interview data shown in FIG. 7.

FIG. 9 is a diagram illustrating a flowchart describing the contents of a medical interview support process executed by the healthcare information processing apparatus in the healthcare information processing system shown in FIG. 1.

FIG. 10 is a diagram illustrating an example of the medical interview data of a state in which an additional symptoms data is added to the medical interview data shown in FIG. 8.

FIG. 11 is a diagram illustrating an example of the contents of the medical interview data held by an electronic medical chart apparatus according to the healthcare information processing apparatus shown in FIG. 1.

FIG. 12 is a diagram illustrating a flowchart describing the contents of a follow-up process executed by the healthcare information processing apparatus in the healthcare information processing system shown in FIG. 1.

FIG. 13 is a block diagram describing an example of the entire configuration of the healthcare information processing system according to a second embodiment.

FIG. 14 is a block diagram illustrating an example of an internal structure of the healthcare information server according to a second embodiment.

### DETAILED DESCRIPTION

With reference to the drawings below, embodiments of a healthcare information processing system will be described. Note that, in the description below, same reference signs are given for components substantially identical in terms of configuration and function, and duplicate description will be given only when necessary.

### First Embodiment

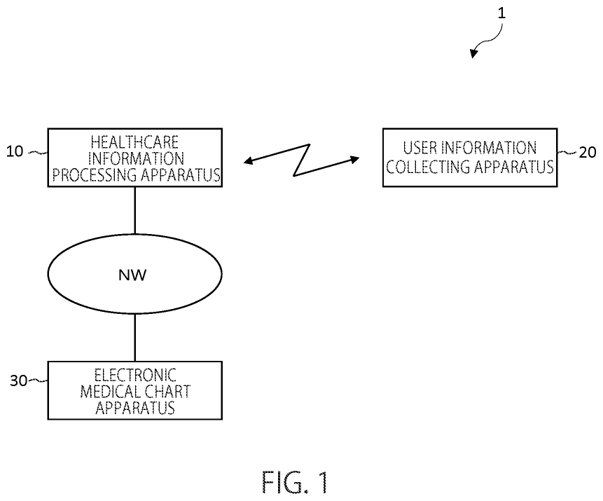

FIG. 1 is a block diagram describing an example of an entire configuration of a healthcare information processing system 1 according to a first embodiment. As shown in FIG. 1, the healthcare information processing system 1 according to the present embodiment, for example, is configured comprising a healthcare information processing apparatus 10, a user information collecting apparatus 20, and an electronic medical chart apparatus 30.

The healthcare information processing apparatus 10 is an apparatus which collects a healthcare information of a user from the user information collecting apparatus 20 and processes data of the collected healthcare information. In the present embodiment, the healthcare information processing apparatus 10 and the user information collecting apparatus 20, for example, may be connected via wireless communication such as Wireless Fidelity (wi-fi), or otherwise may be connected via Local Area Network (LAN) which is also called as a home network. In the present embodiment, the user is a subject for which the healthcare information is collected, and is also appropriately called as a patient when assumed to have some physical symptoms.

The user information collecting apparatus 20 is an apparatus which measures and collects the healthcare information of the user. Specifically, in the present embodiment, the user information collecting apparatus 20 senses and collects at least one information among a vital information, an activity information, and an environmental information of the user as healthcare information. That is to say, in the present embodiment, the healthcare information not only includes a physical information of the user, but also the information which possibly will indirectly or directly affect a body of the user. The user information collecting apparatus 20, for instance, may be configured by a smartphone owned by the user, or may be configured by a wearable computer wearable by the user.

Although the healthcare information processing apparatus 10 and the user information collecting apparatus 20 are provided separately in the example of FIG. 1, these two apparatuses may be established as one. For instance, the healthcare information processing apparatus 10 and the user information collecting apparatus 20 may be provided as an embedded system for small devices wearable on an arm or hip of the user. In this case, there is no communication required between the healthcare information processing apparatus 10 and the user information collecting apparatus 20.

The electronic medical chart apparatus 30 is an apparatus which electronically manages a medical chart provided at a medical institution such as hospitals or clinics. The electronic medical chart apparatus 30 and the healthcare information processing apparatus 10, for example, may be connected via a public line such as an internet, or may be connected via a private line such as a wide-area ethernet. In the present embodiment, it is configured such that medical workers such as a doctor in the medical institution may not only access the medical chart managed by the electronic medical chart apparatus 30, but also may transmit data from the healthcare information processing apparatus 10 which collects the healthcare information of the user to the electronic medical chart apparatus 30.

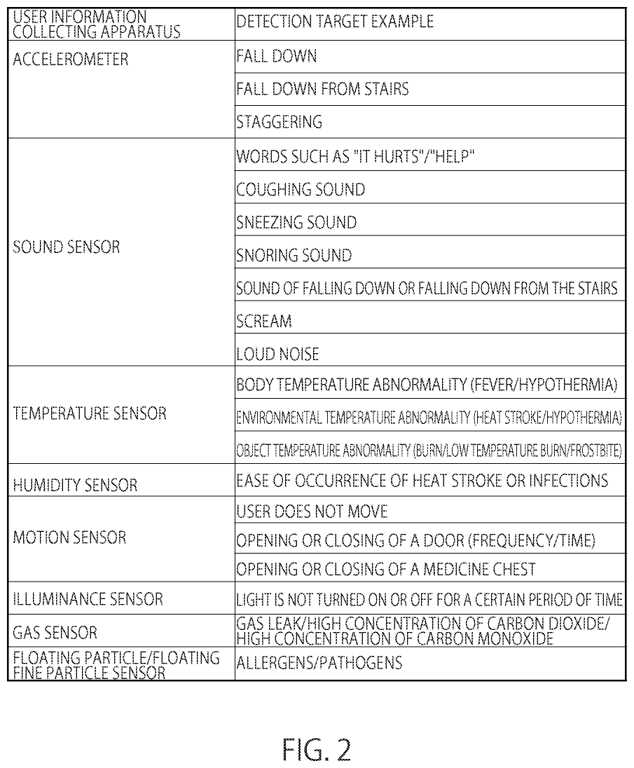

FIG. 2 is a diagram illustrating an exemplary sensor, in a list, which the user information collecting apparatus 20 shown in FIG. 1 may comprise. As shown in FIG. 2, the user information collecting apparatus 20, for example, may be configured comprising at least one sensor among an accelerometer, a sound sensor, a temperature sensor, a humidity sensor, a motion sensor, an illuminance sensor, a gas sensor, and a floating particle/floating fine particle sensor.

The accelerometer may be configured by a wearable sensor worn on the arm or hip of the user. Then, the accelerometer, for instance, detects that the user has fallen down, that the user has fallen down from a stair, or that the user has staggered while standing.

The sound sensor, for instance, may be configured by a voice input apparatus such as a microphone. The sound sensor may be provided here and there in a house of the user, or otherwise may be worn on the arm or hip of the user as the wearable sensor. The sound sensor, for instance, may detect words of the user such as “It hurts” or “Help,” detect a coughing sound of the user, detect a sneezing sound of the user, detect a snoring sound of the user, detect a sound by the user falling down, detect a scream of the user, or detect a loud noise made by the user.

The temperature sensor, for instance, may be configured by a non-contact type sensor such as an infrared sensor, or may be configured by a contact type sensor using a thermistor etc. The non-contact type temperature sensor, for example, may be provided here and there in the house of the user. On the other hand, the contact type temperature sensor may be worn on a wrist of the user as the wearable sensor. The temperature sensor, for example, may detect body temperature abnormalities such as a fever or a hypothermia of the user, may detect environmental temperature abnormalities which becomes a cause for a heat stroke or the hypothermia etc., and may detect object temperature abnormalities such as a burn, a low temperature burn, or a frostbite.

The humidity sensor, for instance, may be configured by a sensor which detects a moist absorption and dehumidification of a moisture sensitive material as a change of electrical resistance or capacity. For instance, an ease of occurrence of the heat stroke or infections may be determined by detecting humidity around the user.

The motion sensor, for instance, may be configured by a camera provided here and there in the house of the user. Then, the motion sensor, for instance, by analyzing images captured by the camera, may detect an event in which the user does not move, detect an event in which a door opens or closes, detect an event in which a frequency of the door opening or closing per hour changes significantly, and detects an event in which a medicine chest opens or closes.

The illuminance sensor, for instance, may be configured by a photodiode provided here and there in the house of the user. Then, the illuminance sensor, for instance, may detect an event in which light in the house of the user is turned on for a certain period of time without being turned off or not turned on for a certain period of time.

The gas sensor, for instance, may be configured by an electrochemical detector with a precious metal catalyst between a pair of electrodes. The gas sensor, for instance, may be provided in a kitchen of the house of the user, or may be provided here and there in the house of the user. Then, the gas sensor, for instance, may detect a gas leak, detect a high concentration of carbon dioxide, or detect a high concentration of carbon monoxide.

The floating particle/floating fine particle sensor, for instance, may be configured by an apparatus which irradiates a detection target, air, with laser light and detects that scattered light. The floating particle/floating fine particle sensor, for example, may be provided here and there in the house of the user. Then, by using the floating particle/floating fine particle sensor to measure an amount or density of a floating particle/floating fine particle in the air, for instance, although indirectly, a presence or amount of allergens and pathogens may be estimated.

Note that each sensors described above are only examples of sensors which the user information collecting apparatus 20 may comprise. For this reason, the user information collecting apparatus 20 may comprise sensors other than types described above. In other words, the healthcare information related to the user collected by the user information collecting apparatus 20 is not limited to the types described above. For instance, a smart home making use of AI or Internet of Things (IoT) technology are implemented in recent years. When the smart home is applied in the house of the user, the healthcare information related to the user may be collected using facilities of this smart home.

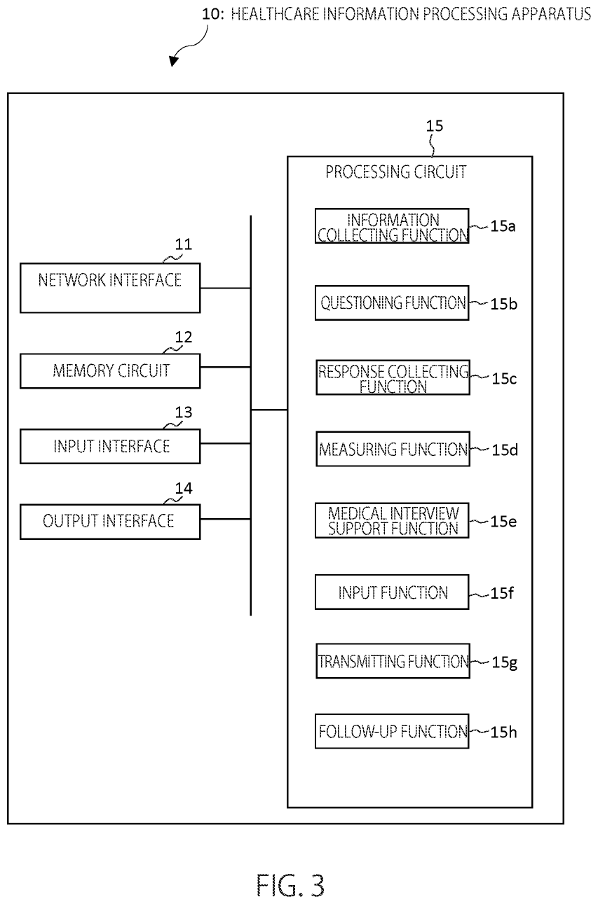

FIG. 3 is a block diagram illustrating an example of an inner configuration of the healthcare information processing apparatus 10 in the healthcare information processing system 1 shown in FIG. 1. As shown in FIG. 3, the healthcare information processing apparatus 10 according to the present embodiment, for example, is configured comprising a network interface 11, a memory circuit 12, an input interface 13, an output interface 14, a processing circuit 15, which are connected to each other via an internal bus.

The network interface 11 controls a transmission and communication of various data performed between the user information collecting apparatus 20 and the electronic medical chart apparatus 30 based on a command from the processing circuit 15. For instance, the network interface 11 receives various information from the user information collecting apparatus 20 and the electronic medical chart apparatus 30 and outputs the received information to the processing circuit 15. This network interface 11, for example, may be realized by a network card, a network adapter, or a NIC (network interface controller) etc.

The memory circuit 12 stores various data or program based on the command from the processing circuit 15. For example, the memory circuit 12 is realized by a RAM (Random Access Memory), a semiconductor memory element such as a flash memory, a hard disk, or an optical disk etc. This memory circuit 12 configures a memory means according to the present embodiment.

The input interface 13 converts an input operation received from the user into an electrical signal and outputs to the processing circuit 15. For instance, the input interface 13 is realized by an input apparatus such as a trackball, a switch button, a mouse, a keyboard, a touchpad which performs input operations by touching an operating surface, a touch screen in which a display screen and a touchpad is integrated, a non-contact input interface using an optical sensor, or the voice input interface. Note that the input interface 13 may be a control circuit such as a connecting interface which receives the electrical signal corresponding to the operation from an operating apparatus provided separately from the healthcare information processing system 1 or the healthcare information processing apparatus 10.

Likewise, the input interface 13 may be configured by the voice input apparatus such as the microphone. When the input interface 13 is configured by the voice input apparatus, the voice input apparatus detects sounds made by the user or sounds made by a user activity, converts into the electrical signal, and outputs to the processing circuit 15.

The output interface 14 outputs various information from the processing circuit 15. The output interface 14, for instance, may be configured by a display. When the output interface 14 is configured by the display, for instance, the output interface 14 is realized by display apparatus such as an Organic EL (Electro Luminescence) monitor, a Liquid Crystal Display, a CRT (Cathode Ray Tube) monitor, or a touch panel. The display, for instance, may display a GUI (Graphical User Interface) to receive user instructions, various image data for display, or various processing results from the processing circuit 15. When the display comprises the GUI to receive user instructions, the display functions as a part of the input interface 13.

Likewise, the output interface 14 may be configured by a voice output apparatus such as a speaker. When the output interface 14 is configured by the voice output apparatus, the output interface 14 outputs various sounds based on the command from the processing circuit 15 and conveys to the user.

The processing circuit 15 controls each component element of the healthcare information processing apparatus 10. For example, the processing circuit 15 is realized by a processor. Especially, in the present embodiment, the processing circuit 15 has an information collecting function 15a, a questioning function 15b, a response collecting function 15c, a measuring function 15d, a medical interview support function 15e, an input function 15f, a transmitting function 15g, and a follow-up function 15h.

Here, for example, each processing function executed by the information collecting function 15a, the questioning function 15b, the response collecting function 15c, the measuring function 15d, the medical interview support function 15e, the input function 15f, the transmitting function 15g, and the follow-up function 15h which are the component elements in the processing circuit 15 shown in FIG. 3, are stored in the memory circuit 12 in a form of computer executable program. The processing circuit 15 is a processor which realizes functions that correspond to each program by reading and executing each program from the memory circuit 12. In other words, the processing circuit 15 in a state in which each program is read has each function shown in the processing circuit 15 of FIG. 3.

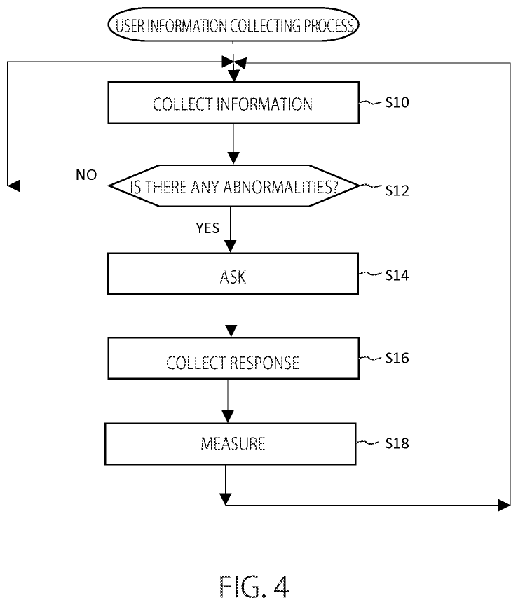

Next, the user information collection process executed by the healthcare information processing apparatus 10 of the healthcare information processing system 1 according to the present embodiment will be described based on FIG. 4. The user information collecting process shown in FIG. 4 is a process regularly executed in the healthcare information processing apparatus 10.

As shown in FIG. 4, the healthcare information processing apparatus 10 first collects the healthcare information of the user from the user information collecting apparatus 20, associates with date or time, and causes the memory circuit 12 to store healthcare information of the user (Step 10). Specifically, the information collecting function 15a in the processing circuit 15 of the healthcare information processing apparatus 10 collects at least one information among the vital information, the activity information, and the environmental information from the user information collecting apparatus 20, associates with a date or time, and causes the memory circuit 12 to store it. Specific example of these vital information, activity information, and environmental information are as described using FIG. 2. For instance, in the present embodiment, information such as a body temperature, a coughing sound, a sneezing sound, a snoring sound, or staggering configures the vital information; information such as falling down, falling down from the stairs, opening or closing of the door, or opening or closing of the medicine chest configures the activity information; and information such as environmental temperature, object temperature, humidity, or gas leak comprises the environmental information.

Likewise, the information collecting function 15a in the processing circuit 15 may be configured to collect information about a drug use of the user as the healthcare information of the user and causes the memory circuit 12 to store it. For instance, when the motion sensor of the user information collecting apparatus 20 detects opening or closing of the medicine chest by the user, the information collecting function 15a in the processing circuit 15 causes the memory circuit 12 to store that the medicine chest was opened or closed. When causing the memory circuit 12 to store the information about drug use, the information collecting function 15a in the processing circuit 15 may add a date and time information and/or a location information in which the drug has been used and may chronologically causes the memory circuit 12 to store it.

Further, the information collecting function 15a in the processing circuit 15 may be configured to further collect information which identifies the drug used by the user as the healthcare information of the user and store in the memory circuit 12. For instance, the motion sensor of the user information collecting apparatus 20 records an image of the drug which the user has taken out from the medicine chest, and the information collecting function 15a in the processing circuit 15 analyzes the image recording the drug, identifies the drug taken out by the user, and causes the memory circuit 12 to store the identified information. When identifying the drug, a product name of that drug may be identified. Likewise, when identifying the drug, types of the drug such as a draft of medicine, over-the counter drug, and supplements may be identified. When causing the memory circuit 12 to store information identifying the drug used by the user, the date and time information and/or the location information which the drug was identified may be added and chronologically stored in the memory circuit 12.

Likewise, for example, there is an IOT medication management system (https://monoistatmarkit.co.jp/mn/articles/1707/26/news024. html) as a system which identifies drugs. This is a system that stores cartridges provided with an IC tag, in the medicine chest with a communication function and manages drugs taken out from the medicine chest by the user. The information collecting function 15a in the processing circuit 15 may identify the type of drug which the user has taken out from the medicine chest by using such medication management system. The information collecting function 15a which executes these various functions configures an information collecting means according to the present embodiment.

Next, as shown in FIG. 4, the healthcare information processing apparatus 10 analyzes the healthcare information of the user collected at Step S10 and determines whether any abnormality has occurred to the user (Step S12). Specifically, the questioning function 15b in the processing circuit 15 of the healthcare information processing apparatus 10, based on the collected healthcare information, determines whether a predefined condition which judges that abnormality has occurred to the user was met. For instance, when the user said “It hurts” or when the body temperature of the user exceeds 37 degrees, the questioning function 15b in the processing circuit 15 determines that the predefined condition which judges that abnormality has occurred to the user was met.

Likewise, in the present embodiment, for instance, the questioning function 15b of the healthcare information processing apparatus 10 may comprise AI, and this AI may monitor the user on a daily basis. Then, the AI determines whether abnormality has occurred to the user based on the voice, gesture, or sound of the user. Likewise, the AI may determine that abnormality has occurred to the user when detecting that the user fell down or fell down the stairs. Furthermore, the AI may determine a change of a body condition of the user as an abnormality based on the vital information.

At Step S12, when determining that no abnormality has occurred to the user (Step S12: No), the healthcare information processing apparatus 10 standbys, repeating the process from Step S10 described above.

On the other hand, when determining that abnormality has occurred to the user (Step S12: Yes), the healthcare information processing apparatus 10 asks a question to the user (Step S14). Specifically, the questioning function 15b in the processing circuit 15 of the healthcare information processing apparatus 10 asks a question to the user. For instance, the questioning function 15b in the processing circuit 15 calls out to the user, “Is something wrong?” from the voice output apparatus, which is a part of the output interface 14, or from the user information collecting apparatus 20, asking the user for changes in body condition. The reason for asking the question is because, in many cases, the healthcare information processing apparatus 10 cannot collect necessary information since, typically, the user does not tend to speak out about personal changes. The questioning function 15b which executes these various processes configures a questioning means according to the present embodiment.

Next, the healthcare information processing apparatus 10 collects a user response to the question of Step S14 and causes the memory circuit 12 to store it (Step S16). Specifically, the response collecting function 15c in the processing circuit 15 of the healthcare information processing apparatus 10 causes the memory circuit 12 to store the user response. In the present embodiment, the response collecting function 15c in the processing circuit 15, for example, collects the response via the voice of the user from the microphone which is a part of the input interface 13 or from the user information collecting apparatus 20, converts into a character data with voice mining, and causes the memory circuit 12 to store it.

Likewise, the response collecting function 15c in the processing circuit 15, when causing the memory circuit 12 to store the user response, may associate the response to the questions with the healthcare information collected by the information collecting function 15a and causes the memory circuit 12 to store it. Especially in the present embodiment, the response collecting function 15c in the processing circuit 15 adds the date and time information and/or the location information to the user response and chronologically causes the memory circuit 12 to store it. These healthcare information and the user response to the questions are desirable to be stored in the memory circuit 12 as a dataset of a same date and time. The response collecting function 15c which executes these various processes configures a response collecting means according to the present embodiment. Likewise, in the present embodiment, the data including the healthcare information and the response to the questions are appropriately called as collected data.

Note that, when detecting that the user has opened or closed the medicine chest, this may be determined as an occurrence of a type of abnormality at Step S12, and the user may be questioned at Step 14. That is to say, the questioning function 15b in the processing circuit 15 may call out to the user, “Is something wrong?” from the voice output apparatus, which is the part of the output interface 14 or from the user information collecting apparatus 20 and may ask why the user has opened or closed the medicine chest or why the user has taken the drug to the user. The user response is stored in the memory circuit 12 by the response collecting function 15c of the processing circuit 15 at Step S16. This user response is also associated with the healthcare information, added the date and time information and/or the location information, and chronologically stored in the memory circuit 12. By this, a change in the body condition of the user may be recorded with more detail.

Next, as shown in FIG. 4, the healthcare information processing apparatus 10 measures a frequency of event that has occurred to the user based on the healthcare information and/or response to the questions stored in the memory circuit 12 (Step S18). Specifically, the measuring function 15d in the processing circuit 15 of the healthcare information processing apparatus 10 measures the event that has occurred to the user based on the healthcare information and/or the response to the questions. The frequency of the measured events, for instance, is stored in the memory circuit 12 by the measuring function 15d in the processing circuit 15.

In the present embodiment, for instance, the measuring function 15d in the processing circuit 15 measures an occurrence frequency of predefined symptoms of the user based on the healthcare information and/or response to the questions stored in the memory circuit 12. For example, when there are multiple responses of “my head hurts” in the user response to the questions, it measures the occurrence frequency of headache. The measured frequency, for example, is expressed as a measurement result such as 5 times per day. Then, the measuring function 15d in the processing circuit 15 causes the memory circuit 12 to store the measured frequency. The measuring function 15d at this time configures a first measuring means according to the present embodiment.

Likewise, for instance, the measuring function 15d in the processing circuit 15 may measure a start date and time of use and a frequency of use of the drug used by the user, based on the healthcare information and/or response to the question stored in the memory circuit 12. For example, based on the information about the type of drug taken by the user, which is included in the healthcare information, it measures the start time and date of use and the frequency of use of taking an analgesic for headaches. For example, the measurement result is expressed such that the start time of taking the drug is Aug. 1, 2021, and the frequency of use is 3 times a day. Then, the measuring function 15d in the processing circuit 15 causes the memory circuit 12 to store the start date and time of the drug used by the user and the measured frequency of use. The measuring function 15d at this time configures a second measuring means according to the present embodiment.

After the process of Step S18, the healthcare information processing apparatus 10 returns to Step S10 described above and repeats the process from Step S10. For this reason, the user information collecting process shown in FIG. 4 is a process regularly executed in the healthcare information processing apparatus 10.

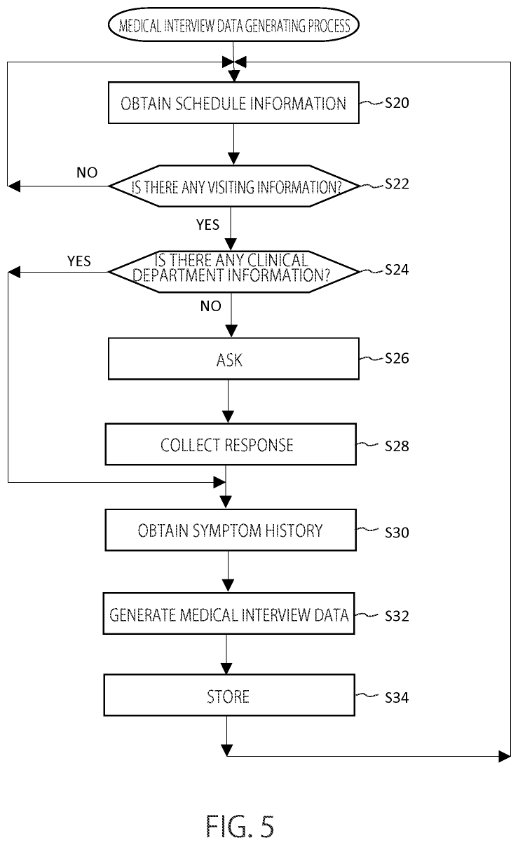

Next, based on FIG. 5, a medical interview data generating process executed by the healthcare information processing apparatus 10 in the healthcare information processing system 1 according to the present embodiment will be described. The medical interview data generating process shown in FIG. 5 is a process regularly executed in the healthcare information processing apparatus 10.

As shown in FIG. 5, the healthcare information processing apparatus 10 first obtains a schedule information of the user (Step S20). Specifically, a medical interview support function 15e in the processing circuit 15 of the healthcare information processing apparatus 10 reads the schedule information of the user from the memory circuit 12 and obtains this. That is to say, in the present embodiment, the user inputs a personal schedule to the healthcare information processing apparatus 10 using the input function which is a part of the input interface 13 in the processing circuit 15 of the healthcare information processing apparatus 10. The healthcare information processing apparatus 10 causes the memory circuit 12 to store the schedule information entered by the user.

The schedule information entered by the user includes various information such as shopping or work. In the present embodiment, this various information includes a visiting information of medical institutions such as the hospital, or information about a clinical department. For example, the schedule information entered by the user includes the information to visit the medical institution of hospital A on August 5, or in some cases, information to visit a neurosurgery department of hospital A.

For this reason, the healthcare information processing apparatus 10 determines whether the schedule information of the user obtained at Step S20 includes the visiting information of the medical institution (Step S22). Specifically, the medical interview support function 15e in the processing circuit 15 of the healthcare information processing apparatus 10 determines whether the information to visit the medical institution is in the schedule information of the user read from the memory circuit 12. When there is no information to visit the medical institution (Step S22: No), it returns to Step S20 described above and standbys, repeating this process.

On the other hand, when information to visit the medical institution is included (Step S22: Yes), it determines whether the clinical department information which the user visits is included in the schedule information of the user obtained at Step S10 (Step S24). Specifically, the medical interview support function 15e in the processing circuit 15 of the healthcare information processing apparatus 10 determines whether the information of the clinical department which the user visits is included in the schedule information of the user read from the memory circuit 12.

Then, when the schedule information of the user does not include the information of the clinical department which the user visits (Step S24: No), the healthcare information processing apparatus 10 asks the user for information about the clinical department which the user plans to visit (Step S26). Specifically, the questioning function 15b in the processing circuit 15 of the healthcare information processing apparatus 10 calls out to the user, “Which clinical department do you plan to visit?” from the voice output apparatus, which is a part of the output interface 14 or from the user information collecting apparatus 20, and asks the user for information about the clinical department to visit.

Next, the healthcare information processing apparatus 10 collects the user response to the questions of Step S26 and, for example, causes the memory circuit 12 to store it (Step S28). Specifically, the response collecting function 15c in the processing circuit 15 of the healthcare information processing apparatus 10 collects the user response and causes the memory circuit 12 to store it. For instance, if the user responds, “I plan to visit the neurosurgery department”, the response collecting function 15c causes the memory circuit 12 to store that the clinical department to visit is the neurosurgery department as the schedule information of the user. As described above, in the present embodiment, for instance, it collects the response via the voice of the user from the microphone, which is a part of the input interface 13, converts into the character data with voice mining, and causes the memory circuit 12 to store it. Of course, the user may input the response to the question as the character data using the keyboard which is a part of the input interface 13. Likewise, if the user information collecting apparatus 20 comprises a microphone, the response collecting function 15c in the processing circuit 15 may collect the response via the voice of the user from the user information collecting apparatus 20.

After Step S28, or when the information of the clinical department which the user visits is included in the schedule information of the user at Step S24 (Step S24: Yes), the healthcare information processing apparatus 10 obtains a history information about the symptoms of the user (Step S30). Specifically, the medical interview support function 15e in the processing circuit 15 of the healthcare information processing apparatus 10 reads the collected data including the healthcare information of the user or the response to the questions from the memory circuit 12, extracts information about the symptoms of the user, and generates the history information about the symptoms. The history information about the symptoms, for instance, includes chronological information to notice the change in the body condition of the user or information about the drug used by the user.

Next, the healthcare information processing apparatus 10 extracts necessary information from the history information about the symptoms of the user and generates the medical interview data (Step S32). Specifically, the medical interview support function 15e in the processing circuit 15 of the healthcare information processing apparatus 10 extracts the history information relevant to the clinical department which the user plans to visit, among the history information about the symptoms of the user obtained at Step S30, and generates the medical interview data. This is to prevent the medical interview being excessive information to the doctor.

For instance, if the user who has a daily headache and a lower back pain visits orthopedics as the clinical department, it extracts history information about the lower back pain among the history information about the symptoms of the user and generates the medical interview data. However, at Step S32, the medical interview data may be generated from the history information about the symptoms of the user without narrowing down the information. This is because, for instance, a toothache may occur in a case of heart attack, and there also are opinions that, information about the symptoms are not to be narrowed down when considering such symptoms.

Likewise, the medical interview data may include the date and time which the symptom had first occurred. For instance, the medical interview support function 15e in the processing circuit 15 may analyze the history information about the symptoms of the user, identify the date and time which that symptom has first occurred, and include that date and time in the medical interview data.

Furthermore, the medical interview data may include an occurrence frequency of symptoms measured by the measuring function 15d in the processing circuit 15. For instance, the medical interview support function 15e in the processing circuit 15 may read the occurrence frequency of symptoms stored by the measurement function 15d in the memory circuit 12 and may include the read occurrence frequency of symptoms in the medical interview data.

Likewise, the medical interview data may include a time which the symptom is likely to occur. For instance, the medical interview support function 15e in the processing circuit 15 may analyze the history information about the symptoms of the user, may identify the time which the symptom is likely to occur as a trend, and may include the identified time in the medical interview data.

Note that the information included in the medical interview data described here are merely examples, and that, in addition to these information or instead of these information, other information may be included in the medical interview data.

Next, the healthcare information processing apparatus 10 stores the medical interview data generated at Step S32 (Step S34). Specifically, the medical interview support function 15e in the processing circuit 15 of the healthcare information processing apparatus 10 stores the generated medical interview data in the electronic medical chart apparatus 30. For instance, the measuring function 15d in the processing circuit 15 transmits the generated medical interview data via a network NW, and the electronic medical chart apparatus 30 receives the medical interview data, and stores the medical interview data in a patient database in association with the information identifying the user.

Note that the medical interview data may let the user input a doctor notification information which is the symptom that the user wishes to convey to the doctor and/or information about the drugs used, via the input interface 13 etc. Specifically, the input function 15f in the processing circuit 15 of the healthcare information processing apparatus 10 cause the user to additionally input symptoms which the user wishes to convey to the doctor or the drugs personally has used. The input function 15f at this time configures an input means according to the present embodiment.

Then, the input doctor notification information is transmitted to the electronic medical chart apparatus 30 of the medical institution via the network interface 11 and the network NW. Specifically, the transmitting function 15g in the processing circuit 15 of the healthcare information processing apparatus 10 transmits the input doctor notification information to the electronic medical chart apparatus 30. The transmitting function 15g at this time configures a first transmitting means according to the present embodiment. The transmitted doctor notification information is added to the medical interview data held by the electronic medical chart apparatus 30.

After the process of Step S34, the healthcare information processing apparatus 10 returns to Step S20 described above and repeats the process from this Step S20. For this reason, the medical interview data generating process shown in FIG. 5 is a process regularly executed in the healthcare information processing apparatus 10.

Note that timings to execute the process of Steps S30 to S34 are arbitrary. For instance, the user may identify the date to visit the medical institution based on the schedule information of the user obtained at Step S20, and the medical interview data may be generated by executing the process of Steps S30 to S34 on a morning of that identified date to visit. Otherwise, the medical interview data may be updated until visiting the medical institution by regularly executing Steps S30 to S34.

Next, the process actually executed by the user information collecting process of FIG. 4 described above and the medical interview data generating process of FIG. 5 will be chronologically described with an example in the case where the user bruises a head.

In this example, suppose that the user bruises the head by falling down in a bedroom of the house, 11 days before visiting the medical institution. The date is Sep. 1, 2020. Since the accelerometer of the user information collecting apparatus 20 equipped by the user has reacted, the healthcare information processing apparatus 10 has asked the user “Is something wrong?” If the user replies “I fell down and bruised my head on a desk,” the healthcare information processing apparatus 10 associates the healthcare information with an acceleration data measured by the accelerometer at the time of falling down, the contents of user response, and the date and time information and the location information of falling down, and cause the memory circuit 12 to store.

Since a lump on the head hurt that day, the user decide to apply a poultice by oneself and opened the medicine chest at the living room of the house. Then, the motion sensor in the user information collecting apparatus 20 detects opening or closing of the medicine chest, and cause the memory circuit 12 to store with the date and time information and the location information when the medicine chest is opened or closed as the healthcare information.

Likewise, the healthcare information processing apparatus 10 asks the user, “Which medicine did you use?” The user responds “Poultice” to this question. In this case, the healthcare information processing apparatus 10 may further ask the user, “Is something wrong?” for a reason of the drug use. If the user responds, “I fell down and got a lump on my head,” it associates the date and time information and the location information with the healthcare information to the user response to the question, and stores in the memory circuit 12.

Since the user also used the poultice on the next day, September 2, by the healthcare information processing apparatus 10, with the same procedures as procedures described above, the healthcare information and the response to the questions are associated and stored in the memory circuit 12 with the time and date information and the location information.

On the next day, September 3, the user used an eye drop due to an eye strain. Then, the healthcare information processing apparatus 10, with the same procedures as described above, associates the information of using the eye drop with the healthcare information and stores in the memory circuit 12.

Furthermore, an analgesic was used at the house on September 9, at the house and office on September 10, and at the office on September 11. In this example, it is assumed that the motion sensor of the wearable user information collecting apparatus 20 worn by the user makes it possible to detect the user taking the drug even at the office.

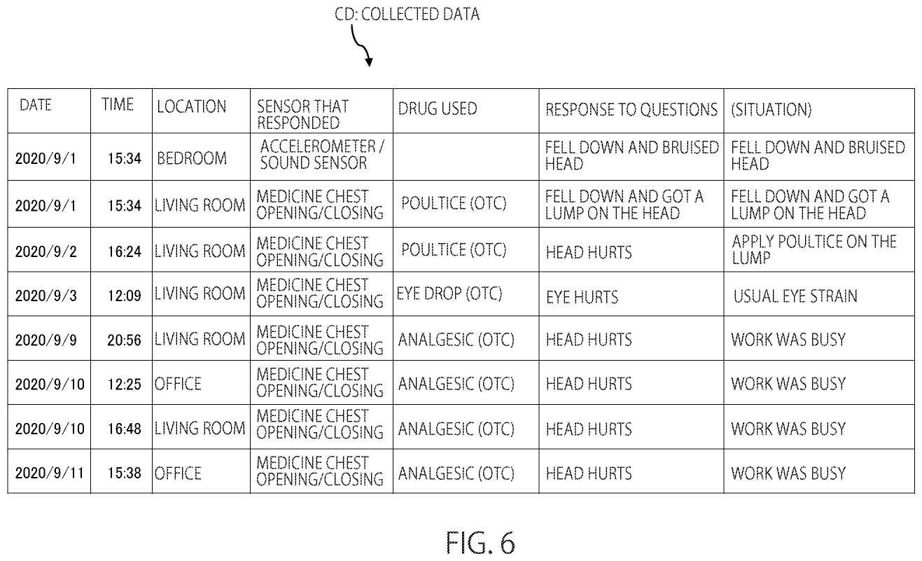

When such series of events occur, the healthcare information processing apparatus 10 generates a collected data CD which associates the healthcare information such as shown in FIG. 6 with the user response to the questions, and causes the memory circuit 12 to store this collected data CD. Note that a situation column shown in FIG. 6 is a description for convenience to describe the event that has occurred to the user, which is not actually included in the collected data CD stored in the memory circuit 12 of the healthcare information processing apparatus 10.

As shown in FIG. 6, during three days from September 9 to September 11, the user plans to visit the medical institution, worried that the headache continues. However, the user forgets bruising the head 11 days ago and believes that the headache has come from busy work, makes an appointment to visit an internal department, and registers that schedule in the healthcare information processing apparatus 10.

The healthcare information processing apparatus 10, detecting that the user plans to visit the ‘internal department’, extracts the history information of symptoms relevant to the ‘internal department’ of the clinical department, and generates the medical interview data.

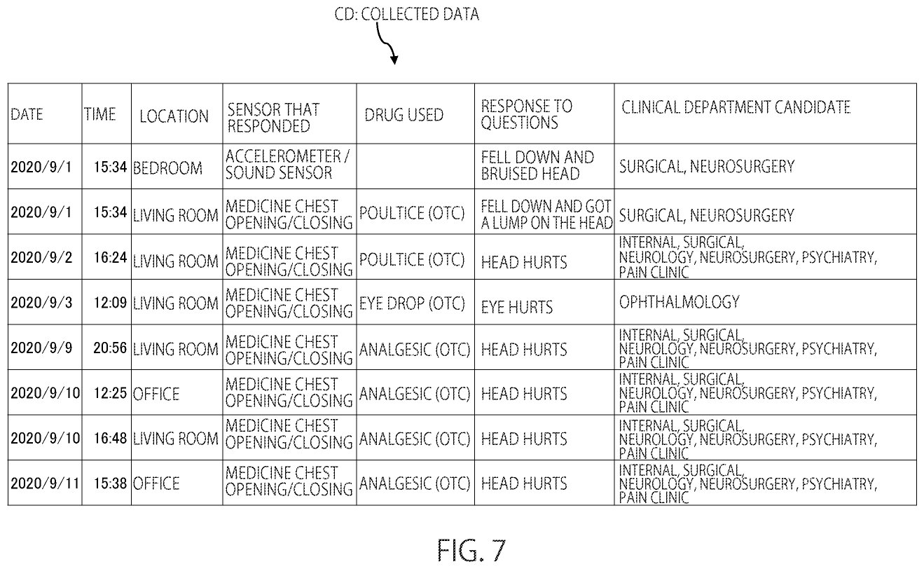

FIG. 7 is a diagram describing an example of a mechanism where the healthcare information processing apparatus 10 according to the present embodiment using the collected data CD extracts the history information of symptoms relevant to the clinical department which the user plans to visit. In this example of FIG. 7, the AI of the healthcare information processing apparatus 10 generates a clinical department candidate data for every occurred event, indicating a candidate of which clinical department the user should visit when visiting the medical institution and adds to the collected data CD.

For example, for an event where the accelerometer and the sound sensor react and where the response to the question is “I fell down and bruised my head,” the clinical department candidate data of “surgical/neurosurgical department” is generated, which is stored in the memory circuit 12 as the collected data CD added to this event. Likewise, for an event where the opening or closing of the medicine chest is detected and where the response to the question is “My head hurts,” the clinical department candidate data of “internal/surgical/neurology/neurosurgery/psychiatry/pain clinic department” is generated, which is stored in the memory circuit 12 as the collected data CD added to this event.

When detecting the history information of the symptom relevant to the clinical department which the user plans to visit, the history information of symptoms to be extracted is determined based on the clinical department candidate data stored in the collected data CD. For instance, if the clinical department which the user plans to visit is “internal department,” the history information of the event including “internal department” in the clinical department candidate data is extracted, and the medical interview data is generated.

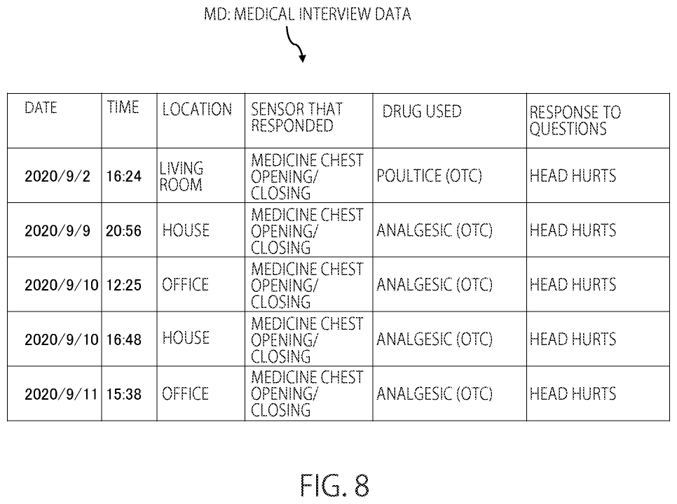

FIG. 8 is a diagram illustrating an example of the medical interview data MD extracted as such. As shown in FIG. 8, since that the clinical department is the “internal department,” the history information of symptoms necessary for visiting the internal department is extracted, and the medical interview data MD is generated. That is to say, bruising the head is an information not included in the medical interview data MD to visit the internal department.

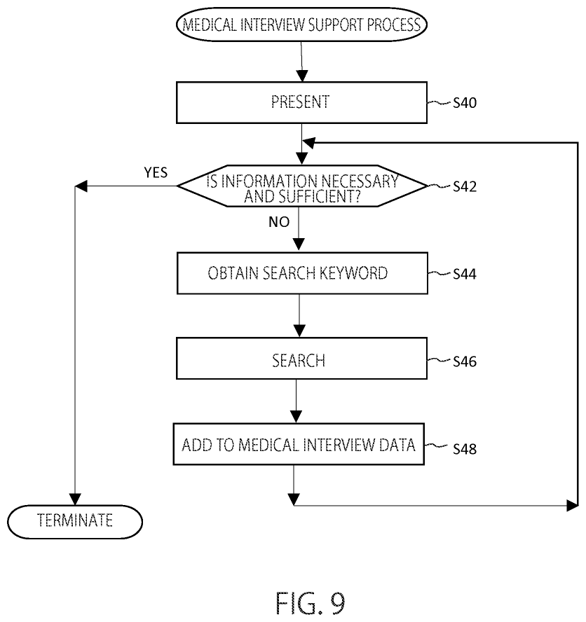

Next, based on FIG. 9, the medical interview support process executed by the healthcare information processing apparatus 10 of the healthcare information processing system 1 according to the present embodiment will be described. The medical interview support process shown in FIG. 9 is a process executed in a case when the user responds to the medical interview from the doctor. For instance, in the embodiment, when the user inputs a start of the medical interview to the healthcare information processing apparatus 10 via the input interface 13 to the healthcare information processing apparatus 10, this medical interview support process starts up.

As shown in FIG. 9, the healthcare information processing apparatus 10 first presents the collected data CD including the healthcare information of the user stored in the memory circuit 12 and the user response to an associated question (Step S40). Specifically, the medical interview support function 15e in the processing circuit 15 of the healthcare information processing apparatus 10 presents the collected data CD including the healthcare information of the user stored in the memory circuit 12 and the user response to the question associated with this healthcare information. By this, for instance, the doctor is presented with the healthcare information and the user response to the question as shown in FIG. 6. This presentation, for instance, is realized by displaying on the display which is a part of the output interface 14 in the healthcare information 10 of the user. Otherwise, this may be realized by the collected data CD including the healthcare information and the response to the question being transmitted from the healthcare information processing apparatus 10 to the electronic medical chart apparatus 30 and presented on the display comprised by the electronic medical chart apparatus 30. The medical interview support function 15e at this time configures a first medical interview support means according to the present embodiment.

Also, the doctor also confirms the medical interview data MD presented by the electronic medical chart apparatus 30 if necessary. That is to say, the doctor confirms the medical interview data MD generated by the medical interview data generating process described above. In this case, the electronic medical chart apparatus 30, for instance, displays on the display the medical interview data MD of FIG. 8 generated in the medical interview data generating process. Note that the medical interview data MD may be presented by the healthcare information processing apparatus 10 instead of the electronic medical chart apparatus 30. As such, the doctor collects information necessary for diagnosing the user based on the healthcare information of the user and the response to the question presented by the healthcare information processing apparatus 10, or based on the medical interview data MD presented by the electronic medical chart apparatus 30.

Note that, at Step S40, the medical interview support function 15e in the processing circuit 15 of the healthcare information processing apparatus 10 may extract and present the information about the drug used by the user among the collected data CD stored in the memory circuit 12. The medical interview support function 15e at this time comprises a second medical interview support means according to the present embodiment.

Likewise, at Step S40, the medical interview support function 15e in the processing circuit 15 of the healthcare information processing apparatus 10 may select and present relevant information among the collected data CD stored in the memory circuit 12 based on a purpose of the user visiting the medical institution. For instance, when the purpose of the user visiting the medical institution is dizziness, the healthcare information and the response to the question related to dizziness are selected and presented. The medical interview support function 15e at this time configures a third medical interview support means according to the present embodiment.

Likewise, at Step S40, the medical interview support function 15e in the processing circuit 15 of the healthcare information processing apparatus 10 may select and present relevant information among the information related to the drug use of the user stored in the memory circuit 12 based on the purpose of the user visiting the medical institution. For instance, if the purpose of the user visiting the medical institution is headache, the information about drug use related to headache is selected and presented. The medical interview support function 15e at this time configures a fourth medical interview support means according to the present embodiment.

Next, the doctor determines whether enough information necessary to diagnose or examine is obtained in the information presented at Step S40 (Step S42). In the present embodiment, for instance, the user inputs via the input interface 13 whether the necessary and sufficient information is obtained or not. Otherwise, the doctor inputs to the electronic medical chart apparatus 30 whether the necessary and sufficient information is obtained or not, and this input information is transmitted to the healthcare information processing apparatus 10. When necessary and sufficient information for the doctor to diagnose or examine is obtained (Step S42: Yes), the medical interview support process terminates.

On the other hand, when necessary and sufficient information for the doctor to diagnose or examine is not obtained (Step S42: No), the healthcare information processing apparatus 10 obtains a search keyword to search from the collected data CD including the healthcare information and the response to the questions (Step S44). Specifically, the medical interview support function 15e in the processing circuit 15 of the healthcare information processing apparatus 10 obtains the search keyword input by the doctor or the user via the input interface 13. For instance, the input via the doctor or the user is done by the keyboard or the voice input apparatus which is a part of the input interface 13. Likewise, the input of the search keyword may be done by the keyboard comprised by the electronic medical chart apparatus 30. In this case, the search keyword input by the doctor to the electronic medical chart apparatus 30 is transmitted to the healthcare information processing apparatus 10 via the network NW.

However, a diagnostic AI may identify the search keyword instead of identifying the search keyword by an experience of the doctor diagnosing the user. For instance, if the diagnostic AI is mounted on the electronic medical chart apparatus 30, the diagnostic AI searches a symptom database held by the diagnostic AI based on the medical interview data MD, and derives search keywords for other possible causes which symptoms identified in the medical interview data MD occur. For example, if the diagnostic AI determines from the medical interview data MD of the user that the headache of the user has suddenly occurred within a few days, it is identified from the symptom database that the search keyword should be surgical causes such as bruising the head.

Next, the healthcare information processing apparatus 10 searches the collected data CD including the healthcare information and the response to the questions based on the search keyword obtained at Step S44, and obtains an additional symptoms data (Step S46). Specifically, the medical interview support function 15e in the processing circuit 15 of the healthcare information processing apparatus 10 searches the collected data CD including the healthcare information and the response to the questions stored in the memory circuit 12 using the search keyword and obtains the additional symptoms data. By this, the healthcare information and the response to the questions relevant to the search keyword are extracted.

Next, the healthcare information processing apparatus 10 adds the additional symptoms data searched at Step S46 to the medical interview data MD (Step S48). Specifically, the medical interview support function 15e in the processing circuit 15 of the healthcare information processing apparatus 10 transmits the extracted additional symptoms data to the electronic medical chart apparatus 30 via the network NW, and the electronic medical chart apparatus 30 adds the received additional symptoms data to the medical interview data MD. By this, the doctor browsing the medical chart in the electronic medical chart apparatus 30 may notice additional symptoms of the user and make it useful for diagnosis or examination.

After Step S48, the healthcare information processing apparatus 10 returns to S42 described above and again repeats from the process determining whether necessary and sufficient information for the doctor to diagnose or examine is obtained. That is to say, this medical information support process is repeated until necessary and sufficient information for the doctor to diagnose or examine is obtained.

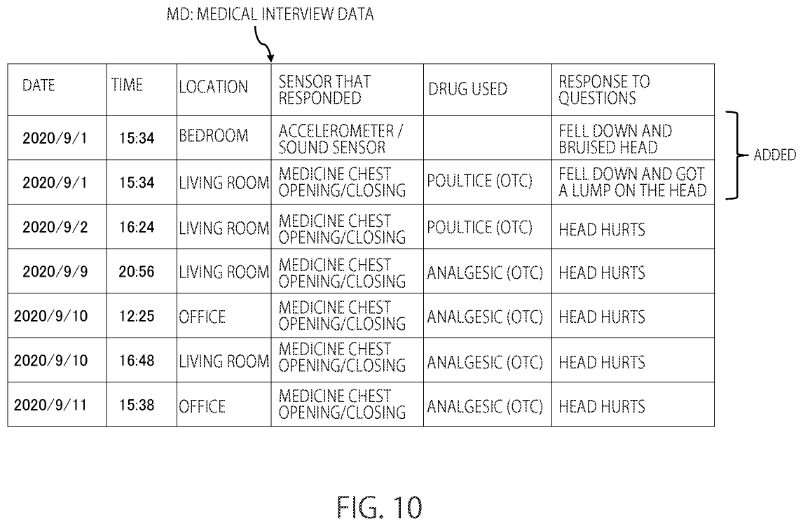

FIG. 10 is a diagram illustrating an example of the medical interview data MD in a state where the additional symptoms data is added to the medical interview data MD shown in FIG. 8. In the example of FIG. 10, for instance, supposing that the doctor inputs “bruising the head” as the search keyword, the healthcare information processing apparatus 10 searches the collected data CD including the healthcare information and the response to the questions stored in the memory circuit 12 and extracts symptoms relevant to “bruising the head.” This extracted data becomes the additional symptoms data and is added to the medical interview data MD held by the electronic medical chart apparatus 30.

When searching based on the search keyword, the search may be done using the search keyword itself, otherwise the search may be done using relevant words of the search keywords in addition to this search keyword or instead of this search keyword. For instance, if the keyword “bruising the head” is input to the electronic medical chart apparatus 30, the diagnostic AI comprised by the electronic medical chart apparatus 30 may identify relevant words of “head” and “bruise,” and may search for collected data CD including the healthcare information and the response to the questions based on these relevant words. By this, a scattering of the search keywords by doctors may be absorbed, and a more appropriate additional symptoms data may be extracted.

The medical interview data MD exemplified in FIG. 10, for instance, is displayed on the display of the electronic medical chart apparatus 30. Then, the doctor may determine to perform a head CT scan to confirm whether the headache of the user is caused by bruising the head. Here, assume that no abnormality caused by bruising the head such as a skull fracture or an intracerebral hemorrhage is found, but a minor abnormality called a straight neck, prone to cause headaches, is found as a result of the head CT scan. For this reason, obtaining from the medical interview data that the headache of the user has occurred during a period while work (PC work) was busy, the doctor determines this as a tension-type headache resulting from straight neck and PC work, and prescribes an analgesic as the draft of medicine.

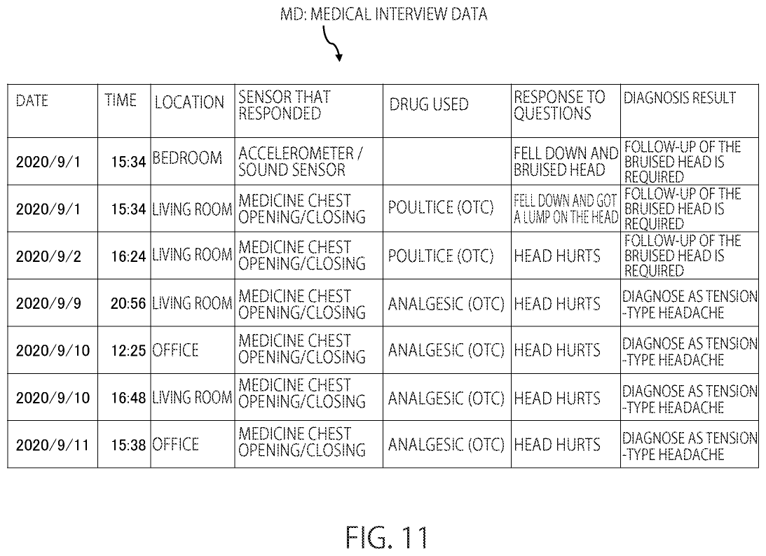

FIG. 11 is a diagram illustrating an example of the contents of the medical interview data MD held by the electronic medical chart apparatus 30 after the diagnosis of the doctor. As shown in FIG. 11, the doctor inputs the diagnosis result to the medical interview data MD after terminating the diagnosis. In the example of FIG. 11, three chronologically oldest events are diagnosed that the bruised head needs a follow-up, and the follow-up requirement is registered in the medical interview data MD. On the other hand, four chronologically new events are diagnosed the tension-type headache, and the tension-type headache is registered in the medical interview data MD.

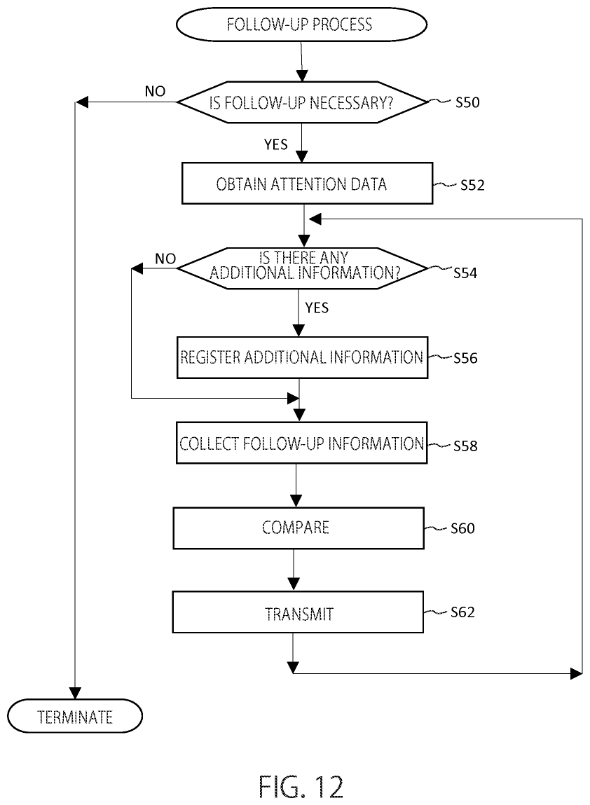

Next, based on FIG. 12, a follow-up process executed by the healthcare information processing apparatus 10 of the healthcare information processing system 1 according to the present embodiment is described. The follow-up process shown in FIG. 12 is a process executed when the diagnosis or examination by the doctor terminates. In the embodiment, for instance, this follow-up process starts up when the doctor or the user inputs the termination of the diagnosis or examination to the healthcare information processing apparatus 10 via the input interface 13 of the healthcare information processing apparatus 10.

As shown in FIG. 12, the healthcare information processing apparatus 10 first determines whether follow-up is necessary or not (Step S50). Specifically, the follow-up function 15h in the processing circuit 15 of the healthcare information processing apparatus 10 inputs whether follow-up is necessary or not via the input interface 13, and based on this input, determines whether the follow-up is necessary or not. Then, if the follow-up function 15h in the processing circuit 15 determines that follow-up is unnecessary (Step S50: No), it terminates this follow-up process.

On the other hand, if the follow-up function 15h in the processing circuit 15 of the healthcare information processing apparatus 10 determines that follow-up is necessary (Step S50: Yes), the healthcare information processing apparatus 10 obtains an attention data (Step S52). Specifically, the follow-up function 15h in the processing circuit 15 of the healthcare information processing apparatus 10 obtains the attention data to identify types of information to pay attention thereafter when following-up the user.

This attention data, for instance, may be input by the doctor to the healthcare information processing apparatus 10 via the input interface 13. Otherwise, the AI comprised by the healthcare information processing apparatus 10 may identify the attention data based on the purpose of the user visiting the medical institution. Likewise, by the doctor inputting the examination result of the user, the AI comprised by the healthcare information processing apparatus 10 may identify the attention data to enhance an observation based on the examination result.

For instance, if the user visits the medical institution by “bruising the head,” follow-up is necessary since there is a rare possibility of a chronic subdural hematoma occurring to the bruised head between about 3 weeks to 6 months after injury. For this reason, suppose that the doctor determines that follow-up is necessary and inputs to the healthcare information processing apparatus 10 that follow-up of the chronic subdural hematoma is necessary. The AI of the healthcare information processing apparatus 10 sets headache, nausea, weakness, and staggering, which are main symptoms of the chronic subdural hematoma, as the attention data based on this input. By this, the healthcare information processing apparatus 10 is set to monitor a frequency of taking analgesics by headache, a frequency of taking gastrointestinal drug by nausea, and a frequency of stumbling caused by weakness or staggering, and to perform the follow-up.

Next, the healthcare information processing apparatus 10 determines (Step S54) whether there is an input of additional information from the user. Specifically, the follow-up function 15h in the processing circuit 15 of the healthcare information processing apparatus 10 determines whether there is the input of additional information from the user. This input of additional information, for instance, is performed by the voice input apparatus or the keyboard which is a part of the input interface 13.

For the medical interview to be precise, things not compassed in the healthcare information processing apparatus 10 may be included, such as symptoms, a past history, or a family history that the user did not convey to the doctor during the medical interview. If the user recalls, this is input to the healthcare information processing apparatus 10 as additional information.

Likewise, the follow-up function 15h in the processing circuit 15 of the healthcare information processing apparatus 10 may ask the user returning home from the medical institution for a “topic of symptom” at an arbitrary timing and may collect the information about a subjective symptom of the user as additional information. Likewise, a conversation between the user and the medical worker other than the doctor who diagnosed the user may be collected as additional information.

When there is the input of additional information (Step S54: Yes), this additional information is registered with the date and time information and/or the location information added, and stored in the memory circuit 12 (Step S56). Specifically, the follow-up function 15h in the processing circuit 15 of the healthcare information processing apparatus 10 converts the user input additional information to the character data etc., adds the date and time information and/or the location information to this, and causes the memory circuit 12 to store as the collected data CD with the healthcare information or the response to the questions.

Note that, when determining that there is no additional information input at Step S54 (Step S54: No), the process of Step S56 is skipped.

Next, the healthcare information processing apparatus 10 collects the follow-up information (Step S58). Specifically, the follow-up function 15h in the processing circuit 15 of the healthcare information processing apparatus 10, from the memory circuit 12, reads the collected data CD stored in the memory circuit 12 obtained after visiting the medical institution and obtains as the follow-up information. This read collected data CD includes the healthcare information, the user response to the questions, and/or additional information. For this reason, the follow-up function 15h in the processing circuit 15 extracts the follow-up information from these healthcare information, user response to the questions, and/or the additional information.

Next, the healthcare information processing apparatus 10 determines whether there is a change that should be notified to the doctor, comparing the follow-up information collected at Step S58 with the healthcare information and the response to the questions before visiting the medical institution (Step S60). Specifically, the follow-up function 15h in the processing circuit 15 of the healthcare information processing apparatus 10 detects a change to the user by chronologically comparing the healthcare information, the response to the questions, and the additional information included in the follow-up information, with the response to the questions and the healthcare information before visiting the medical institution. For instance, the AI of the healthcare information processing apparatus 10 determines whether there is a change in the occurrence frequency of symptoms in the user before and after visiting the medical institution.

Next, the healthcare information processing apparatus 10 transmits the follow-up information collected at Step S58 and/or a comparison result executed at Step S60 to the electronic medical chart apparatus 30 (Step S62). Specifically, the transmitting function 15g in the processing circuit 15 of the healthcare information processing apparatus 10 transmits the follow-up information and/or the comparison result to the electronic medical chart apparatus 30 via the network NW. Whether the follow-up information and/or the comparison result transmitted to the electronic medical chart apparatus 30 is registered or not to an electronic medical chart of the electronic medical chart apparatus 30 is arbitrary. That is to say, this follow-up information and/or the comparison result may be registered to the electronic medical chart of the electronic medical chart apparatus 30, or otherwise, after these follow-up information and/or the comparison result is once stored and held in the electronic medical chart of the electronic medical chart apparatus 30, the doctor may see the follow-up information and/or the comparison result and register in the electronic medical chart based on the doctor's determination.

For this reason, for example, things that the user has forgotten to convey to the doctor or things that the user has felt about the symptoms after visiting the medical institution is registered in the healthcare information processing apparatus 10 as additional information, which may be conveyed to the doctor. Likewise, the doctor may appropriately notice how the occurrence frequency of symptoms after visiting the medical institution changed from before visiting the medical institution. By the doctor referring to this information, the doctor may notice a treatment effect or progress without waiting for the user to revisit.

At Step S62, when determining that symptoms relevant to the purpose of visiting the medical institution has occurred based on the follow-up information and/or the comparison result, the transmitting function 15g in the processing circuit 15 may transmit the occurrence of the symptoms to the electronic medical chart apparatus 30 of the medical institution. In this case, the transmitting function 15g configures a second transmitting means according to the present embodiment. By this, the doctor may see the electronic medical chart apparatus 30 and quickly notice the change of symptoms of the diagnosed patient.

Likewise, at Step S62, when detecting a change in the frequency of use of the drug which the user uses based on the follow-up information and/or the comparison result, the transmitting function 15g in the processing circuit 15 may transmit a change in the frequency of use to the electronic medical chart apparatus 30 of the medical institution. In this case, the transmitting function 15g configures a third transmitting means according to the present embodiment. By this, the doctor may quickly notice the change if the patient forgets to take a prescribed drug or takes it a wrong number of times.

Likewise, at Step S62, when the occurrence frequency of the symptoms in the user exceeds a predefined standard value based on the follow-up information and/or the comparison result, the transmitting function 15g in the processing circuit 15 may transmit a warning to the electronic medical chart apparatus 30 of the medical institution. Likewise, the transmitting function 15g in the processing circuit 15 may transmit the warning to the electronic medical chart apparatus 30 when the frequency of use of the drug used by the user exceeds a predefined standard value. By this, the doctor may quickly notice that an abnormality has occurred to the symptom of the patient based on this warning.

After the process of Step S62, the healthcare information processing apparatus 10 returns to Step S54 described above and repeats the process from this Step S54. For this reason, when determining that follow-up is necessary at Step S50, this follow-up process is a process regularly executed. According to the present embodiment, for instance, the follow-up process shown in FIG. 12 is terminated when the user receives the diagnosis of the doctor again and the doctor inputs to stop the follow-up process to the healthcare information processing apparatus 10.

As described above, in the healthcare information processing system 1 according to the present embodiment, since the healthcare information processing apparatus 10 collects the healthcare information of the user, asks the user when this healthcare information meets a predefined condition, and causes the memory circuit to store the response to the questions, appropriate medical interview data may be easily generated. That is to say, a time when symptoms began to appear, the occurrence frequency of symptoms, and a cause of symptoms may be included in the medical interview data even without the user being conscious.

Likewise, since the healthcare information processing system 1 may also follow-up after visiting the medical institution, the doctor may easily notice the treatment effect etc. by the change of symptoms after the visit, and the doctor may notice the state of the patient quickly and accurately. For this reason, the doctor may develop an appropriate treatment policy by using the healthcare information processing system 1.

### Second Embodiment

Although the healthcare information processing system 1 according to the first embodiment described above causes the memory circuit 12 comprised by the healthcare information processing apparatus 10 to store the collected data CD such as the healthcare information, the response to the questions, and the additional information, a location of storing such information is not limited to the healthcare information processing apparatus 10. In a second embodiment, the collected data CD such as the healthcare information, the response to the questions, and the additional information are stored in the memory circuit of the healthcare information server connected to the network NW. Parts that differ from that of the first embodiment described above will be described.

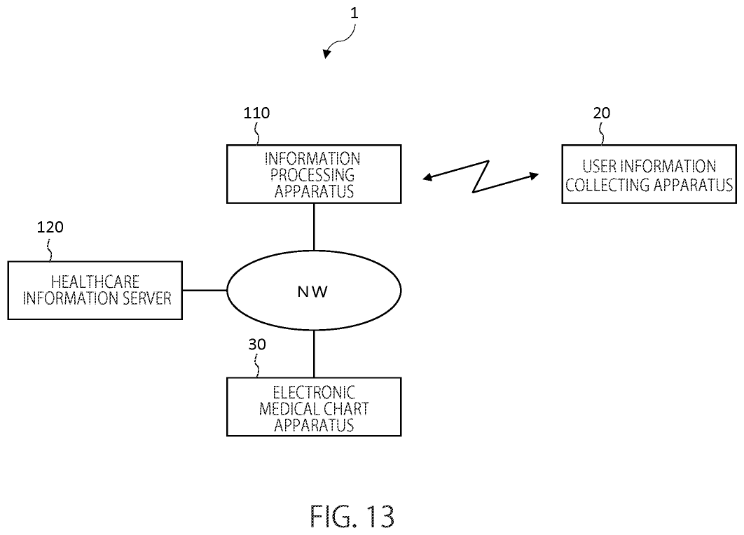

FIG. 13 is a block diagram describing an example of the entire configuration of the healthcare information processing system 1 according to the second embodiment, which corresponds to FIG. 1 of the first embodiment described above. As shown in FIG. 13, in the healthcare information processing system 1 according to the second embodiment, an information processing apparatus 110 and a healthcare information server 120 are connected to the network NW instead of the healthcare information processing apparatus 10. In the example of FIG. 13, the information processing apparatus 110 is an apparatus mounted on each house of the user, and the healthcare information server 120 is an apparatus mounted one for a plurality of information processing apparatuses 110.

As shown in FIG. 13, the information processing apparatus 110 is an apparatus held by the user, which collects healthcare information from the user information collecting apparatus 20. Then, the information processing apparatus 110 communicates with the healthcare information server 120 via the network NW and transmits this healthcare information to the healthcare information server 120.

Likewise, the information processing apparatus 110 comprises a function that asks the user as described above based on the command from the healthcare information server 120. That is to say, the healthcare information server 120 asks the user via the information processing apparatus 110 when the healthcare information meets the predefined condition based on the collected the healthcare information of the user and collects the response to that question. That is to say, the questions to the user are made based on the command from the healthcare information server 120. However, the questions to the user may be made by the information processing apparatus 110 determined by itself, instead of being made by the information processing apparatus 110 based on the command from the healthcare information server 120. Then, the information processing apparatus 110 communicates with the healthcare information server 120 via the network NW and transmits the response to the questions to the healthcare information server 120.

Note that the information processing apparatus 110 and the user information collecting apparatus 20 does not necessarily have to be separate, but may be configured as one. In other words, the information processing apparatus 110 may be omitted by the user information collecting apparatus 20 comprising various functions of the information processing apparatus 110, including a network communication function.

The healthcare information server 120 communicates with the information processing apparatus 110 via the network NW, collects the healthcare information of the user or the user response to the questions, and causes the memory circuit to store. That is to say, the healthcare information server 120 determines whether the healthcare information meets the predefined condition based on the healthcare information obtained from the information processing apparatus 110, and commands the information processing apparatus 110 to ask the user when the predefined condition is met. By this, the information processing apparatus 110 asks the user.

The question to the user may be commanded by the information processing apparatus 110 with the healthcare information server 120 specifying the question. In this case, the information processing apparatus 110 outputs a specified question with voice etc. Otherwise, the question to the user may be commanded by the information processing apparatus 110 without the healthcare information server 120 specifying the question. In this case, the information processing apparatus 110 may output a fixed question of “Is something wrong?” with voice etc., or output an appropriate question with voice etc. based on the healthcare information collected by the information processing apparatus 110.

Then, the healthcare information server 120 communicates with the information processing apparatus 110, receiving and collecting the response to the questions from the information processing apparatus 110. This collected response to the questions are also stored in the memory circuit.

Thereafter, the healthcare information server 120 works together with the information processing apparatus 110 based on the healthcare information or the response to the questions stored in the memory circuit, and executes the user information collecting process, the medical interview data generating process, or the medical interview support process described above.

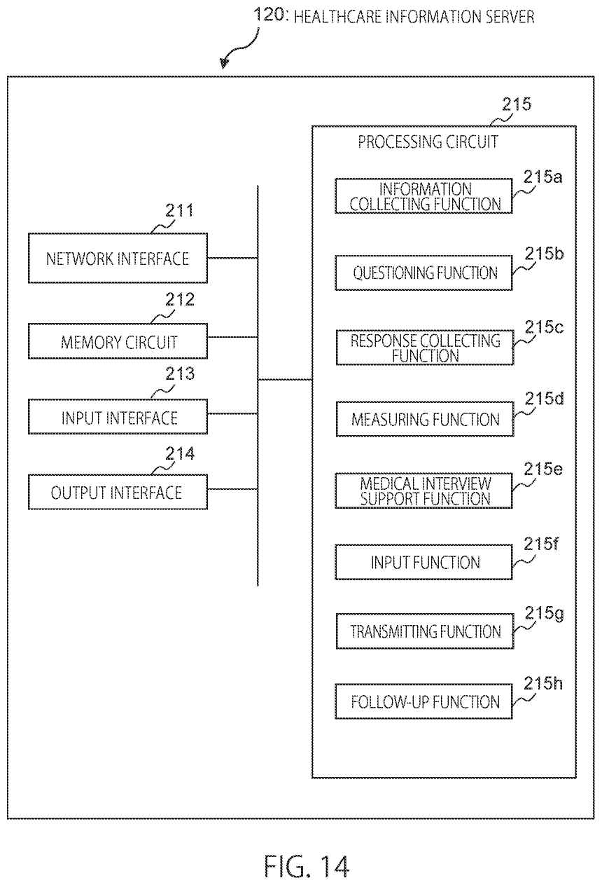

FIG. 14 is a block diagram illustrating an example of an internal structure of the healthcare information server 120 according to the present embodiment, which corresponds to FIG. 3 according to the first embodiment described above. As shown in FIG. 14, the healthcare information server 120 according to the present embodiment, for instance, is configured comprising a network interface 211, a memory circuit 212, an input interface 213, an output interface 214, and a processing circuit 215, each connected via the internal bus. The network interface 211, the memory circuit 212, the input interface 213, the output interface 214, and the processing circuit 215 each correspond to the network interface 11, the memory circuit 12, the input interface 13, the output interface 14, and the processing circuit 15 in the healthcare information processing apparatus 10 according to the first embodiment described above with substantially equivalent functions.

The processing circuit 215 of the healthcare information server 120 comprises an information collecting function 215a, a questioning function 215b, a response collecting function 215c, a measuring function 215d, a medical interview support function 215e, an input function 215f, a transmitting function 215g, and a follow-up function 215h. Generally, the information collecting function 215a, the questioning function 215b, the response collecting function 215c, the measuring function 215d, the medical interview support function 215e, the input function 215f, the transmitting function 215g, and the follow-up function 215h are each equivalent functions to the information collecting function 15a, the questioning function 15b, the response collecting function 15c, the measuring function 15d, the medical interview support function 15e, the input function 15f, the transmitting function 15g, and the follow-up function 15h.

That is to say, in the present embodiment, the healthcare information server 120 not only comprises the main functions of the questioning function 15b and the response collecting function 15c in the processing circuit 15 according to the first embodiment described above, but the healthcare information server 120 also comprises the main functions of the measuring function 15d, the medical interview support function 15e, the transmitting function 15g, and the follow-up function 15h in the processing circuit 15. Compared to this, the main functions of the information collecting function 15a which collects the healthcare information from the user and the input function 15f which is the user input, is comprised by the information processing apparatus 110 in the house of the user.

Specifically, the information collecting function 215a in the processing circuit 215 of the healthcare information server 120 communicates with the information processing apparatus 110 held by the subject, collects at least one information among the vital information, the activity information, and the environmental information of the subject, associate with the date and time, and causes the memory circuit 212 to store it. The questioning function 215b communicates with the information processing apparatus 110 when the collected information meets the predefined condition and commands the information processing apparatus 110 to ask the subject. The response collecting function 215c communicates with the information processing apparatus 110, receives the response to the question from the information processing apparatus 110, and causes the memory circuit 212 to store it.

The measuring function 215d, for instance, measures the event occurred to the subject based on the healthcare information and the response to the questions. A frequency of measured event, for instance, is stored in the memory circuit 212. The medical interview support function 215e, for instance, reads the collected data including the subject healthcare information or the response to the questions from the memory circuit 212. Then, the medical interview support function 215e extracts the information about the symptoms of the subject generates the history information about the symptoms, extracts among this history information the history information relevant to the clinical department which the subject plans to visit, and generates the medical interview data.

The input function 215f, for instance, communicates with the information processing apparatus 110, lets the subject to input information from the information processing apparatus 110, and obtains that input information. The transmitting function 215g, for instance, transmits the doctor notification information obtained by the input function 215f to the electronic medical chart apparatus 30, transmits the follow-up information and/or the comparison result to the electronic medical chart apparatus 30, transmits the occurrence of symptoms relevant to the purpose of visiting the medical institution to the electronic medical chart apparatus 30, or transmits the change of frequency of use of the drug to the electronic medical chart apparatus 30.

The follow-up function 215f, for instance, performs follow-up of the subject and causes the memory circuit 212 to store the additional information when the subject inputs additional information, reads the collected data CD from the memory circuit 212 and generates the follow-up information, or determines whether there is a change that should be notified to the doctor based on this follow-up information.

As described above, in the healthcare information processing system 1 according to the present embodiment, the healthcare information server 120 may work together with the information processing apparatus 110 and collects the healthcare information of the user, may ask the user when this healthcare information meets the predefined condition, and may cause the memory circuit to store the response to the questions. For this reason, even in the healthcare information processing system 1 according to the present embodiment, appropriate medical interview data may be easily generated. That is to say, the time when symptoms began to appear, the occurrence frequency of symptoms, and the cause of symptoms may be included in the medical interview data even without the user being conscious.

Likewise, since the main function of collecting the healthcare information and generating the medical interview data are provided in the healthcare information server 120 such as a web server, the information processing apparatus 110 to be prepared on a user side may be narrowed down to have minimal functions. For this reason, it is possible to configure the information processing apparatus 110 with a laptop PC or the wearable computer.

Note that the word “processor” used in above descriptions means circuits such as, for example, a Central Processing Unit (CPU), a Graphics Processing Unit (GPU), an Application Specific Integrated Circuit (ASIC), a programmable logic device (for example, a Simple Programmable Logic Apparatus (SPLD), a Complex Programmable Logic Apparatus (CPLD), and a Field Programmable Gate Array (FPGA)). The processor executes functions by reading and executing programs stored in the memory. Note that programs may be configured to be directly integrated in the processor instead of being storing in the memory. In this case, the processor realizes functions by reading and executing programs stored in the circuit. Note that the processor is not limited to the case arranged as a single processor circuit, but may be configured as a single processor by combining a plurality of independent circuits to realize functions. Furthermore, a plurality of component elements in FIG. 3 may be integrated into one processor to realize the functions.

While certain embodiments have been described, these embodiments have been presented by way of example only and are not intended to limit the scope of the inventions. The embodiments may be in a variety of other forms. Furthermore, various omissions, substitutions and changes may be made without departing from the spirit of the inventions. The embodiments and their modifications are included in the scope and the subject matter of the invention, and at the same time included in the scope of the claimed inventions and their equivalents.

1. A healthcare information processing apparatus, comprising:
a memory; and
processing circuitry configured to collect at least one information among a vital information, an activity information, and an environment information of a subject, associate with a date and/or time, and store in a memory;
ask questions to the subject when the information meets a predefined condition based on the information of the subject; and
cause the memory to store a response to the questions.

2. The healthcare information processing apparatus of claim 1, wherein an information collector is further configured to collects information about a drug use of the subject as the information of the subject and causes the memory to store.

3. The healthcare information processing apparatus of claim 2, wherein the processing circuitry is further configured to collect information which identifies a drug used by the subject as the information of the subject and causes the memory to store.

4. The healthcare information processing apparatus of claim 1, wherein the processing circuitry is configured to associate the response to the questions with the information collected by an information collector and causes the memory to store.

5. The healthcare information processing apparatus of claim 1, wherein the processing circuitry is further configured to measure an occurrence frequency of predefined symptoms in the subject based on the information stored in the memory and/or the response to the questions.

6. The healthcare information processing apparatus of claim 3, wherein the processing circuitry is further configured to measure a start date and time of use and a frequency of use of the drug used by the subject based on the information stored in the memory and/or the response to the questions.

7. The healthcare information processing apparatus of claim 1, wherein the processing circuitry is further configured to support interviews at a medical institution by presenting the information stored in the memory and/or the response to the questions.

8. The healthcare information processing apparatus of claim 2, wherein the processing circuitry is further configured to support interviews at a medical institution by presenting the information about the drug used by the subject among the information stored in the memory.

9. The healthcare information processing apparatus of claim 1, wherein the processing circuitry is further configured to support interviews at a medical institution by selecting and presenting relevant information among the information stored in the memory and the response to the questions, based on a purpose of the subject visiting the medical institution.

10. The healthcare information processing apparatus of claim 2, wherein the processing circuitry is further configured to support interviews at a medical institution by selecting and presenting relevant information among the information about the drug use of the subject stored in the memory, based on the purpose of the subject visiting the medical institution.

11. The healthcare information processing apparatus of claim 1, wherein the processing circuitry is further configured to
cause the subject to input a doctor notification information about symptoms and/or the drug used by the subject which the subject wishes to convey to a doctor of a medical institution; and
transmit the doctor notification information input by the input to an electronic medical chart apparatus of the medical institution.

12. The healthcare information processing apparatus of claim 1, wherein the processing circuitry is further configured to, after the subject visiting a medical institution, when an information collector and/or a response collector collect that a symptom relevant to the purpose of visiting the medical institution has occurred, transmit the symptom to an electronic medical chart apparatus of the medical institution.

13. The healthcare information processing apparatus of claim 1, wherein the processing circuitry is further configured to, after the subject visiting a medical institution, when detecting a change in a frequency of use of the drug which the subject uses, further transmit the change in the frequency of use to an electronic medical chart apparatus of the medical institution.

14. A healthcare information server, comprising:
a memory; and
processing circuitry configured to
communicate with an information processing apparatus held by a subject, collect at least one information among a vital information, an activity information, and an environment information of the subject, associate with a date and/or time, and store in the memory;
communicate with the information processing apparatus and command the information processing apparatus to ask the subject when the information meets a predefined condition based on the information of the subject; and
communicate with the information processing apparatus, receive a response to the questions from the information processing apparatus, and cause the memory to store the response to the questions.
